(19) United States
(12) Patent Application Publication
(10) Pub. No.: US 2021/0145911 A1
(43) Pub. Date: May 20, 2021

(54) FUNCTIONAL FOOD COMPOSITION FOR ENHANCING MUSCULAR FUNCTION AND MOBILITY COMPRISING LOTUS LEAF EXTRACT, AND METHOD FOR PREPARING SAME

(71) Applicants: COSMAX NS, INC., Gyeonggi-do (KR); COSMAX NBT, INC., Seoul (KR)

(72) Inventors: Jin Hak KIM, Seoul (KR); Ji Hwan JANG, Gyeonggi-do (KR); Jeong Ho GEUM, Seoul (KR); Min Son KWEON, Seoul (KR); Su Young CHOI, Gyeonggi-do (KR); Sang Woo KIM, Gyeonggi-do (KR); Jae Kwan HWANG, Seoul (KR)

(21) Appl. No.: 16/976,376

(22) PCT Filed: Mar. 8, 2019
(86) PCT No.: PCT/KR2019/002737

§ 371 Date: Aug. 27, 2020

(30) Foreign Application Priority Data
Mar. 9, 2018 (KR) 10-2018-0027741

(51) Int. Cl. A61K 36/185; A61P 21/06; A23L 33/00; A23L 33/105

(52) U.S. Cl. CPC A61K 36/185; A61P 21/06; A23L 33/40; A23V 2002/00; A61K 2236/333; A61K 2236/331; A61K 2236/37; A23L 33/105

(57) ABSTRACT

The present invention relates to a functional food composition for enhancing muscular function and exercise performance and a method for preparing same. According to the present invention, provided is a composition which enables increased generation of factors associated with myogenesis and energy metabolism and thus enhances exercise performance and muscular function. More particularly, mitochondria and muscle cell synthesis is increased and muscular atrophy is inhibited, and thus muscle exercise effects can be increased and fatigue is reduced. The composition can be applied as a health function composition to various fields such as foods.

### BACKGROUND OF THE INVENTION

### 1. Field of the Invention

The present invention relates to a functional food composition for enhancing muscular function and exercise performance comprising lotus leaf extract and a method for preparing the same.

### 2. Description of the Related Art

Due to recent development of science and technology and modern medicine, average life expectancy of humans is gradually increasing, and accordingly, interest in health is increasing to improve quality of life. This trend is increasing regardless of generation or age. In addition, as much interest in medicines, dietary supplements, and cosmetics containing natural products increases, research and development thereon are increasing. In particular, the efficacy of natural medicines and the functions of dietary supplements and cosmetics containing natural products are expanding to basic medical scope such as antioxidant, cholesterol suppression, obesity prevention, immunity and disease prevention and aging suppression.

Among natural plants used in natural products, lotus leaves, called “Nelumbinis Folium,” are known to have antimicrobial and blood pressure lowering effects, strengthen stomach, stop bleeding, and release extravasated blood. Lotus leaf has a clean leaf and is known to be good for skin beauty. Among the lotus leaves, the round and large leaves are also known as “Hibiscus mutabilis” as a symbol of beauty.

In addition, the lotus leaves are known to stop diarrhea caused by heat and moisture, relieve thirst, disperse wind and heat accumulated in the head and eyes and relieve dizziness, and be good for treating various bleeding disorders such as hemoptysis, nose bleeding, hematuria and uterine bleeding. It is also known to have antioxidant and detoxifying effects and to help prevent adult diseases caused by free radicals and inhibit aging. In addition, it is also described in the herbal literature that ingestion of lotus leaves helps detoxification when poisoned after eating sea crabs. As the efficacy of these lotus leaves is known, rice is cooked with wrapping in lotus leaves, or lotus leaves are brewed to drink a tea, thereby consuming active ingredients of lotus leaf.

The present inventors have studied substances originating from natural products and found that the composition containing lotus leaf extract has an effect of improving muscular function and exercise performance and thus completed the present invention.

### SUMMARY OF THE INVENTION

An object of the present invention is to provide a functional food composition for enhancing muscular function and exercise performance.

Other object of the present invention is to provide a method for preparing a functional food composition for enhancing muscular function and exercise performance.

In order to solve the above problems, the present invention provides a functional food composition for enhancing muscular function and exercise performance comprising lotus leaf extract.

According to one embodiment, the composition may comprise more than 0% by weight and 100% by weight or less of the lotus leaf extract based on the total weight of the composition.

According to one embodiment, the composition may be a composition for preventing or ameliorating muscle-related diseases and the muscle-related disease may be one or more selected from the group consisting of atony, muscular atrophy, muscular dystrophy, muscle degeneration, myasthenia, myotonia, amyotrophic lateral sclerosis, cachexia and sarcopenia.

In addition, according to other embodiment of the present invention, there is provided a method for preparing a functional food composition for enhancing muscular function and exercise performance, comprising extracting lotus leaves with water, an organic solvent having 1 to 6 carbon atoms or a mixture thereof as a solvent.

In addition, according to one embodiment, there is provided a method for preparing a functional food composition for enhancing muscular function and exercise performance, comprising obtaining lotus leaf extract by subcritical extraction, supercritical fluid extraction or ultrahigh pressure extraction from lotus leaves.

Other specific details of the embodiments of the present invention are included in the following detailed description.

### Effect of the Invention

According to the functional food composition for enhancing muscular function and exercise performance according to the present invention and the method for preparing same, it is possible to provide a composition which enables increased generation of factors associated with myogenesis and energy metabolism and thus enhances exercise performance and muscular function. In addition, the composition can be applied as a health functional composition to various fields such as foods.

### BRIEF DESCRIPTION OF THE DRAWINGS

FIG. 1 is a graph showing the activity of mTOR according to Example 1.

FIG. 2 is a graph showing the activity of PPARδ according to Example 1.

FIG. 3 is a graph showing the activity of PGC-1α according to Example 1.

FIG. 4 is an electrophoresis photograph showing the activity of inhibiting degradation of myoprotein according to Example 1.

FIG. 5 is an electrophoresis photograph showing the activity of mitochondrial biogenesis and myogenesis according to Example 1.

### DETAILED DESCRIPTION OF THE INVENTION

Since various modifications and variations can be made in the present invention, particular embodiments are illustrated in the drawings and will be described in detail in the detailed description. It should be understood, however, that the invention is not intended to be limited to the particular embodiments, but includes all modifications, equivalents, and alternatives falling within the spirit and scope of the invention. In the following description of the present invention, detailed description of known functions will be omitted if it is determined that it may obscure the gist of the present invention.

Hereinafter, a functional food composition for enhancing muscular function and exercise performance according to an embodiment of the present invention and a method for preparing the same will be described in more detail.

As used herein, the term “enhancing muscular function and exercise performance” means improving activities of mTOR, PPARδ and PGC-1α, inhibiting degradation of myoprotein, promoting mitochondrial biogenesis, and promoting myogenesis. Specifically, inhibition of degradation of myoprotein can be confirmed by the decreased mRNA expression of atrogin-1 and MuRF-1. In addition, the activity of promoting mitochondrial biogenesis, and promoting myogenesis can be confirmed by the increased mRNA expression of myogenin, NRF1 and Tfam.

According to one embodiment, it is possible to prevent, treat and ameliorate muscle-related diseases by enhancing muscular function and exercise performance. Prevention, treatment and amelioration of muscle-related diseases can be confirmed by expression of muscle and exercise-related factors such as mTOR, PPARδ and PGC-1α.

The muscle-related disease may include diseases caused by decreased muscle function, muscle wasting, and muscle degeneration. Specifically, diseases such as atony, muscular atrophy, muscular dystrophy, muscle degeneration, myasthenia, myotonia, amyotrophic lateral sclerosis, cachexia and sarcopenia can be prevented, treated, and improved.

The functional food composition for enhancing muscular function and exercise performance according to the present invention comprises lotus leaf extract.

According to one embodiment, the present invention may comprise more than 0% by weight and 100% by weight or less of the lotus leaf extract based on the total weight of the composition, for example 1 to 100% by weight, for example 20 to 70% by weight of the lotus leaf extract.

According to one embodiment, the functional food composition for enhancing muscular function and exercise performance may be prepared by a method comprising extracting lotus leaves with water, an organic solvent having 1 to 6 carbon atoms or a mixture thereof as a solvent to obtain lotus leaf extract. For example, the organic solvent having 1 to 6 carbon atoms may include methanol, ethanol, ethyl acetate, hexane, and the like.

According to one embodiment, the composition according to the present invention can be prepared by a method comprising extracting lotus leaves by ethanol extraction or hot water extraction.

According to an embodiment, in the ethanol extraction, 10 to 95%, for example, 20 to 80%, for example, 50% ethanol may be used. In addition, the ethanol extraction may include extracting by adding ethanol in an amount of 2 to 20 times the volume of the object to be extracted. In addition, for example, the extraction reaction may be performed for 0.5 to 10 hours, for example, 2 to 5 hours, and at 4 to 80° C., for example 20 to 70° C., for example 40 to 60° C.

According to one embodiment, the hot water extraction may include extracting by adding water in an amount of 2 to 20 times the volume of the object to be extracted. In addition, for example, the extraction reaction may be performed for 0.5 to 12 hours, for example, 2 to 6 hours, and at 4 to 121° C., for example 50 to 121° C., for example 80 to 121° C.

According to one embodiment, the functional food composition for enhancing muscular function and exercise performance may be prepared by a method comprising obtaining lotus leaf extract by subcritical extraction, supercritical fluid extraction or ultrahigh pressure extraction from lotus leaves.

Hereinafter, embodiments of the present invention will be described in detail so that those skilled in the art can easily carry out the present invention. The present invention may, however, be embodied in many different forms and should not be construed as limited to the embodiments set forth herein.

### Example 1: Hot Water Extraction

1,500 ml of distilled water was added to 100 g of lotus leaves, and then subjected to hot water extraction at 95° C. for 4 hours using a heating mantle (Extraction Rota-Mantle/KBT) equipped with a reflux cooling device, filtered, concentrated and lyophilized to obtain lotus leaf extract.

### Example 2: Ethanol Extraction

1,500 ml of 10%, 30%, 50%, 70%, 90% ethanol was added to 100 g of lotus leaves, and then subjected to hot water extraction at 70° C. for 4 hours using a heating mantle (Extraction Rota-Mantle/KBT) equipped with a reflux cooling device, filtered, concentrated and lyophilized to obtain lotus leaf extract.

### Example 3: Methanol Extraction

Lotus leaf extract was obtained in the same manner as in Example 2, except that methanol was used.

### Example 4: Ethyl Acetate Extraction

Lotus leaf extract was obtained in the same manner as in Example 2, except that ethyl acetate was used.

### Example 5: Hexane Extraction

Lotus leaf extract was obtained in the same manner as in Example 2, except that hexane was used.

### Example 6: Ultrahigh Pressure Extraction

18% of lotus leaves and 76 mL of ethanol were placed in a polyethylene pack and sealed, and then extracted using an ultrahigh pressure extraction device (Frescal MFP-7000; Mitsubishi Heavy Industries, Tokyo, Japan). The ultrahigh pressure extraction condition was set to an extraction pressure of 320 MPa and an extraction time of 5 minutes. The extracted sample was filtered and concentrated to remove the extraction solvent, and lypophilized to obtain lotus leaf extract.

### Example 7: Supercritical Fluid Extraction

Lotus leaves were filled in a sample cartridge and extracted using a supercritical extraction device (SFX 3560, Isco Inc., Lincoln, Nebr., USA). The supercritical fluid extraction condition was set to an extraction pressure of 20 MPa, an extraction temperature of 60° C., a flow rate of supercritical carbon dioxide of 60 mL/min and an extraction time of 60 minutes. When the supercritical fluid extraction was completed, the pressure of the extraction device was lowered to release the supercritical fluid state, obtaining lotus leaf extract.

### Experimental Example 1: mTOR Activity

The mTOR activity for the extract according to Example 1 was evaluated. mTOR is a protein that plays a role in regulating myoprotein synthesis mechanism, and improvement of muscle mass can be confirmed by confirming the activity of mTOR. First, the muscle parental cell line L6 cells (ATCC) were inoculated and cultured at 2×10⁵ cells/mL in a 6-well plate with a Dulbecco's modified Eagle's media (DMEM; Hyclone) containing 110% fetal bovine serum (FBS; Hyclone, Logan, Utah, USA). When the cell density was about 80 to 85%, the medium in the well was removed and exchanged with DMEM (Hyclone) containing 2% horse serum (HS; Hyclone) to differentiate L6 cells into myotubes. Differentiation was carried out for a total of 6 days by exchanging with a new medium once every 2 days. After differentiation, the extract prepared in Example 1 was dissolved in a DMEM medium containing 50 ng/mL of TNF-α at a concentration of 100 or 200 μg/mL, and then the cells were treated therewith. After 6 hours, total RNA was isolated using a TRIzol reagent (Takara, Osaka, Japan). The isolated total RNA was quantified using Nanodrop (NanoDrop 1000; Thermo Fisher Scientific Inc., Waltham, Mass., USA). The results are shown in Table 1.

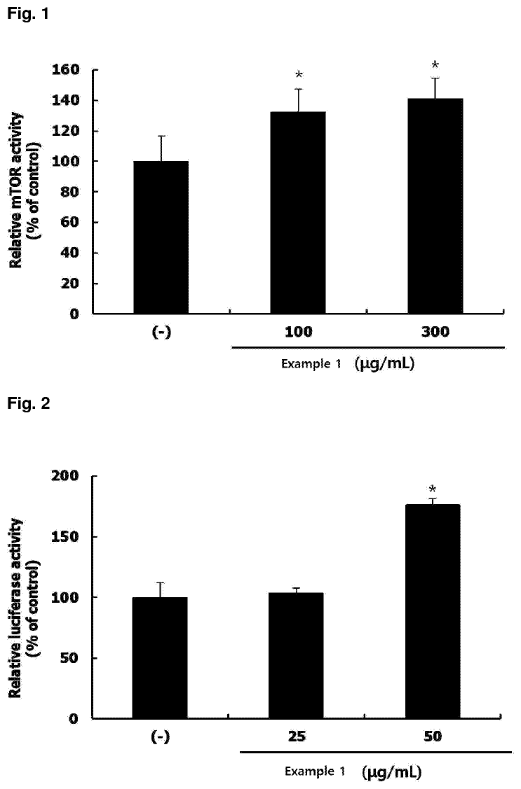

In addition, the cells were treated with the material according to Example 1 at a concentration of 100 or 300 μg/mL, and the results are shown in FIG. 1.

TABLE 1

Concentration (μg/mL)

| Korean name | 100 | 200 |
|---|---|---|
| Lotus leaf | 150.44 ± 1.55* | 166.63 ± 7.92* |

| | |
|---|---|
| Control | 100.00 ± 9.91 |

As shown in Table 1 and FIG. 1, it is found that the growth of myoprotein can be induced by the material according to Example 1 of the present invention.

### Experimental Example 2: PPARδ Activity

In order to evaluate the PPARδ activity for the extract according to Example 1, COST cells (ATCC) were placed in a DMEM medium containing 10% FBS (Gibco) at 1×10⁵ cells/mL in a 24-well plate. When the cell density was about 80 to 85%, transformation of PPARδ was induced using Lipofector EXT (Aptabio) in order to verify the anti-inflammatory effect. After transformation, the cells were treated with each extract at a concentration of 10 μg/mL for 24 hours. And luciferase activity was evaluated using a luciferase assay kit (Promega).

PPARδ is a protein that can be associated with cell differentiation in adipose tissue and sugar production in liver tissue, and the improvement of exercise performance can be confirmed by confirming the activity of PPARδ. The results are shown in Table 2.

In addition, the cells were treated with the material according to Example 1 at a concentration of 25 or 50 μg/mL, and the results are shown in FIG. 2.

TABLE 2

Concentration (μg/mL)

| Korean name | 25 | 50 |
|---|---|---|
| Lotus leaf | 97.72 ± 6.90 | 115.44 ± 20.62* |

| | |
|---|---|
| Control | 100.00 ± 5.72 |

As shown in Table 2 and FIG. 2, it is found that the exercise performance can be improved by the material according to Example 1 of the present invention.

### Experimental Example 3: PGC-1α Activity

In order to evaluate the PGC-1α activity for the extract according to Example 1, COST cells (ATCC) were placed in a DMEM medium containing 10% FBS (Gibco) at 1×10⁵ cells/mL in a 24-well plate. When the cell density was about 80 to 85%, transformation of peroxisome proliferator-activated receptor gamma coactivator-1 alpha (PGC-1α) was induced using Lipofector EXT (Aptabio) in order to verify the anti-inflammatory effect. After transformation, the cells were treated with each extract at a concentration of 10 μg/mL for 24 hours. And luciferase activity was evaluated using a luciferase assay kit (Promega).

PGC-1α is a protein associated with sugar regulation and mitochondrial function replication, and the improvement of exercise performance can be confirmed by confirming the activity of PGC-1α. The results are shown in Table 3.

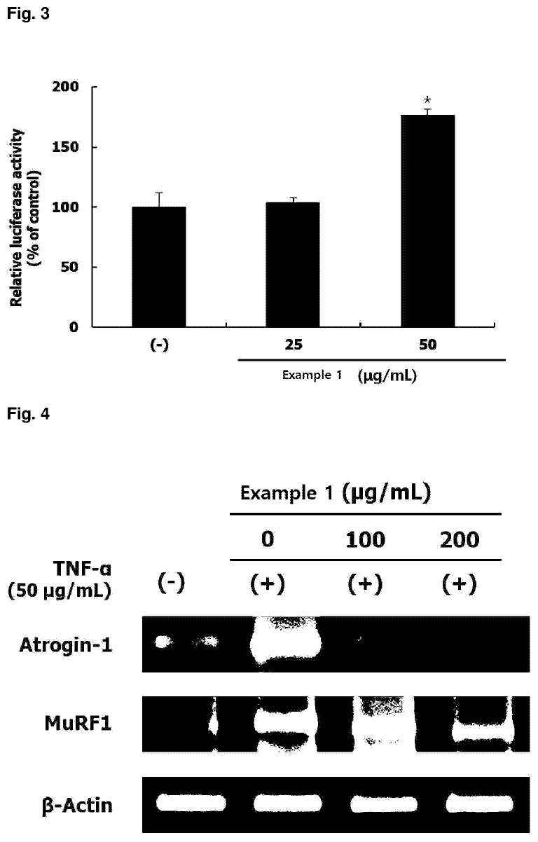

In addition, the cells were treated with the material according to Example 1 at a concentration of 25 or 50 μg/mL, and the results are shown in FIG. 3.

TABLE 3

Concentration (μg/mL)

| Korean name | 25 | 50 |
|---|---|---|
| Lotus leaf | 103.99 ± 4.05 | 176.59 ± 5.17′ |

| | |
|---|---|
| Control | 100.00 ± 11.99 |

As shown in Table 3 and FIG. 3, it is found that the exercise performance can be improved by the material according to Example 1 of the present invention.

### Experimental Example 4: Activity of Inhibiting Degradation of Myoprotein

In order to confirm the activity of inhibiting degradation of myoprotein for the extract according to Example 1, expression of atrogin-1, MuRF1 and β-actin was confirmed. Atrogin-1 is a muscle destructing protein expressed during muscle atrophy, MuRF1 is a muscle degrading protein in muscle, and β-actin is a protein that makes up muscles and causes muscle contraction and relaxation along with myosin.

First, the muscle parental cell line L6 cells (ATCC) were inoculated and cultured at 2×10⁵ cells/mL in a 6-well plate with Dulbecco's modified Eagle's media (DMEM; Hyclone) containing 110% fetal bovine serum (FBS; Hyclone, Logan, Utah, USA). When the cell density was about 80 to 85%, the medium in the well was removed and exchanged with DMEM (Hyclone) containing 2% horse serum (HS; Hyclone) to differentiate L6 cells into myotubes. Differentiation was carried out for a total of 6 days by exchanging with a new medium once every 2 days. After differentiation, the lotus leaf extract prepared in Example 1 was dissolved in a DMEM medium containing 50 ng/mL of TNF-α at a concentration of 100 or 200 μg/mL, and then the cells were treated therewith. After 6 hours, total RNA was isolated using a TRIzol reagent (Takara, Osaka, Japan). The isolated total RNA was quantified using Nanodrop (NanoDrop 1000; Thermo Fisher Scientific Inc., Waltham, Mass., USA). 16 μL RNA was quantified and mixed with Reverse Transcriptase Premix (ELPIS-Biotech), and synthesized to cDNA at 42° C. for 55 minutes and at 70° C. for 15 minutes using a PCR machine (Gene Amp PCR System 2700; Applied Biosystems, Foster City, Calif., USA). 4 μL of cDNA of the synthesized cDNA, the following specific primer pair (Bioneer, Deajeon, Korea) and PCR premix (ELPIS-Biotech) were mixed to prepare a PCR sample and then PCR was repeated 30 times under conditions of 95° C. for 30 seconds, 60° C. for 1 minute and 72° C. for 1 minute.

The amplified cDNA as a result of PCR was separated by electrophoresis with a 1.5% agarose gel, and cDNA bands were identified using a G:BOX EF imaging system (Syngene, Cambridge, UK). The sequences used are shown in Table 4, and the results are shown in FIG. 4.

TABLE 4

| Item | Forward primer | Reverse primer |
|---|---|---|
| Atrogin-1 | 5′-CCCTGAGTGGC | 5′-AGGTCCCGCCC |
| | ATCGCCCAA-3′ | ATCGCTCA-3′ |
| MuRF-1 | 5′-GAAATGCTATG | 5′-ATTCCTGCTTA |
| | CAGAACCTG-3′ | GATGTCG-3′ |
| β-Actin | 5′-AGCCATGTACG | 5′-CTCTCAGCTGT |
| | TAGCCATCC-3′ | GGTGCTGAA-3′ |

As shown in FIG. 4, it is found that the mRNA expression of atrogin-1 and MuRF-1 is decreased in L6 muscle cells as treated with the lotus leaf extract. This means that the lotus leaf extract of the present invention is excellent in inhibiting degradation of myoprotein in muscle cells.

### Experimental Example 5: Activity of Promoting Mitochondrial Biogenesis and Promoting Myogenesis

In order to confirm the activity of promoting muscle mitochondrial biogenesis and promoting myogenesis for the extract according to Example 1, expression of myogenin, NRF1 and Tfam was confirmed. Myogenin controls differentiation and maturation into myotubes, NRF1 is involved in mitochondrial protein production, and Tfam is involved in mitochondrial DNA transcription.

First, the muscle parental cell line L6 cells (ATCC) were inoculated and cultured at 2×10⁵ cells/mL in a 6-well plate with Dulbecco's modified Eagle's media (DMEM; Hyclone) containing 110% fetal bovine serum (FBS; Hyclone, Logan, Utah, USA). When the cell density was about 80 to 85%, the medium in the well was removed and the lotus leaf extract prepared in Example 1 was dissolved in a DMEM medium containing 2% HS (Hyclone) at a concentration of 100 or 200 μg/mL, then the cells were treated therewith to induce differentiation into myotubes. At this time, the group treated with 0.01% DMSO instead of the sample was used as a control. This process was repeated 3 times of 2 days for a total of 6 days to induce differentiation, and then total RNA was isolated using a TRIzol reagent (Takara, Osaka, Japan). The isolated total RNA was quantified using Nanodrop (NanoDrop 1000; Thermo Fisher Scientific Inc., Waltham, Mass., USA). 16 μL RNA was quantified and mixed with Reverse Transcriptase Premix (ELPIS-Biotech), and synthesized to cDNA at 42° C. for 55 minutes and at 70° C. for 15 minutes using a PCR machine (Gene Amp PCR System 2700; Applied Biosystems, Foster City, Calif., USA). 4 μL of cDNA of the synthesized cDNA, the following specific primer pair (Bioneer, Deajeon, Korea) and PCR premix (ELPIS-Biotech) were mixed to prepare a PCR sample and then PCR was repeated 30 times under conditions of 95° C. for 30 seconds, 60° C. for 1 minute and 72° C. for 1 minute.

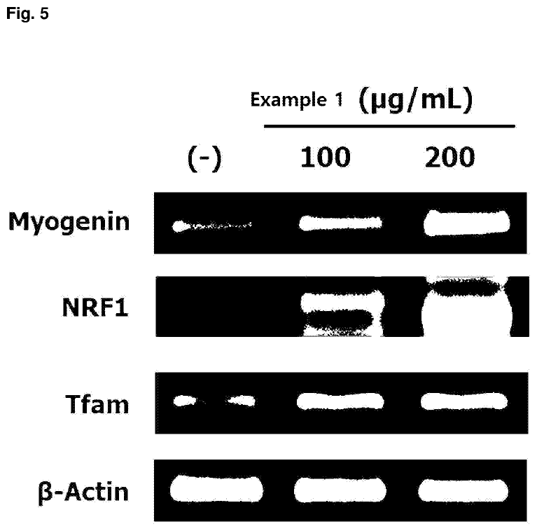

The amplified cDNA as a result of PCR was separated by electrophoresis with a 1.5% agarose gel, and cDNA bands were identified using a G:BOX EF imaging system (Syngene, Cambridge, UK). The sequences used are shown in Table 5, and the results are shown in FIG. 5.

TABLE 5

| Item | Forward primer | Reverse primer |
|---|---|---|
| Myogenin | 5′-TGGGCTGCCA | 5′-CAGCCCAGCC |
| | CAAGCCAGAC-3′ | ACTGGCATCA-3′ |
| NRF1 | 5′-TGGACCCAAG | 5′-GGTCATTTCA |
| | CATTACGGAC-3′ | CCGCCCTGTA-3′ |
| Tfam | 5′-GCTTCCAGGA | 5′-CCCAATCCCA |
| | GGCTAAGGAT-3′ | ATGAGAACTC-3′ |
| β-Actin | 5′-CTGTGTCTGAT | 5′-GGTAAAACGC |
| | TTTGGCTCTAT-3′ | AGCTCAGTAACA-3′ |

As shown in FIG. 5, it is found that the mRNA expression of myogenin, NRF1 and Tfam is increased in L6 muscle cells as treated with the lotus leaf extract according to Example 1. This means that the lotus leaf extract of the present invention is excellent in promoting mitochondrial biogenesis and promoting myogenic differentiation in muscle cells.

As described above, the results according to Experimental Examples 1 to 5 are summarized in Tables 6 and 7.

TABLE 6

| | |
|---|---|
| Muscle mass | Exercise |

| Korean name | mTOR | PPARδ | PGC-1α |
|---|---|---|---|
| Lotus leaf | ◯ | ◯ | ◯ |

TABLE 7

| | | Endurance | | | |
|---|---|---|---|---|---|
| | | Luciferase | | Muscle strength | |
| Scientific name | Sample | activity assay | RT-PCR | ELISA Kit | RT-PCR |
| Nelumbo nucifera | Lotus | PGC-1^{(increase)} | NRF-1^{(increase)} | mTOR^{(increase)} | Myogenin^{(increase)} |
| Gaertner | leaf | PPARδ^{(increase)} | Tfam^{(increase)} | | MuRF1^{(increase)} |
| | | | | | Atrogin-1^{(increase)} |

As can be seen from the above results, it is found that according to the present invention mitochondria and muscle cell synthesis is increased and muscular atrophy is inhibited, and thus muscle exercise effects can be increased and fatigue is reduced.

The above description is merely illustrative of the technical spirit of the present invention, and those skilled in the art to which the present invention pertains can make various modifications and variations without departing from the essential characteristics of the present invention. In addition, the embodiments disclosed in the present invention are not for limiting the technical spirit of the present invention but for illustration, and the scope of the technical spirit of the present invention is not limited by these embodiments. The scope of protection of the present invention should be interpreted by the claims appended hereto, and all technical spirits within the scope equivalent thereto should be interpreted as being included in the scope of the present invention.

What is claimed is:

1. A functional food composition for enhancing muscular function and exercise performance comprising lotus leaf extract.

2. The functional food composition for enhancing muscular function and exercise performance according to claim 1, wherein the composition comprises more than 0% by weight and 100% by weight or less of the lotus leaf extract based on the total weight of the composition.

3. The functional food composition for enhancing muscular function and exercise performance according to claim 1, wherein the food composition is a composition for preventing or ameliorating muscle-related diseases and the muscle-related diseases is one or more selected from the group consisting of atony, muscular atrophy, muscular dystrophy, muscle degeneration, myasthenia, myotonia, amyotrophic lateral sclerosis, cachexia and sarcopenia.

4. A method for preparing a functional food composition for enhancing muscular function and exercise performance comprising lotus leaf extract, the method comprising extracting lotus leaves with water, an organic solvent having 1 to 6 carbon atoms or a mixture thereof as a solvent to obtain lotus leaf extract.

5. A method for preparing a functional food composition for enhancing muscular function and exercise performance, comprising obtaining lotus leaf extract by subcritical extraction, supercritical fluid extraction or ultrahigh pressure extraction from lotus leaves.
